(19) United States
(12) Patent Application Publication
(10) Pub. No.: US 2026/0325821 A1
(43) Pub. Date: Oct. 22, 2026

(54) Integration of Pulmonary Artery Pressure, Systemic Blood Pressure, and LVAD Pump Parameters

(71) Applicant: TC1 LLC, St. Paul, MN (US)

(72) Inventors: Daniel I. Harjes, Carlisle, MA (US); Chad Dague, Windham, NH (US); Pritika Toutam, Plymouth, MN (US)

(73) Assignee: TC1 LLC, St. Paul, MN (US)

(21) Appl. No.: 19/695,476

(22) Filed: Jun. 2, 2026

Related U.S. Application Data
(63) Continuation of application No. PCT/US2024/058067, filed on Dec. 2, 2024.
(60) Provisional application No. 63/607,174, filed on Dec. 7, 2023.

(51) Int. Cl. A61M 60/17; A61M 60/592; A61M 60/90

(52) U.S. Cl. CPC A61M 60/17; A61M 60/592; A61M 60/90; A61M 2205/04; A61M 2205/18; A61M 2205/3303; A61M 2205/3327; A61M 2205/3334; A61M 2205/3344; A61M 2205/3365; A61M 2205/3523; A61M 2205/3592; A61M 2205/502; A61M 2210/125; A61M 2230/005; A61M 2230/04

(57) ABSTRACT

Systems and related methods for remotely managing care of a patient with an implanted left ventricular assist device are provided. A system A system for managing care of a patient having an implanted left ventricular assist device (LVAD) and an implanted pulmonary artery pressure (PAP) sensor includes a patient management server and a clinician portal. The patient management server is configured to receive PAP data and LVAD data for a patient. The PAP data is indicative of PAP of the patient measured via a PAP sensor implanted in the patient. The LVAD data is indicative of measured operational parameters of an LVAD implanted in the patient. The clinician portal is configured to communicate an alert to the clinician in response to at least one of the PAP data and the LVAD data being indicative of a change in a hemodynamic condition of the patient.

### CROSS-REFERENCE TO RELATED APPLICATIONS

The present application is a Continuation of PCT/US2024/058067 filed Dec. 2, 2024; which claims the benefit of U.S. Provisional Patent Application No. 63/607,174 filed Dec. 7, 2023, the full disclosures which are incorporated herein by reference in their entirety for all purposes.

### BACKGROUND

Ventricular assist devices, known as VADs, are used for both short-term (i.e., days, months) and long-term blood circulation assistance (i.e., years or a lifetime) where a patient's heart is incapable of providing adequate circulation, commonly referred to as heart failure or congestive heart failure. According to the American Heart Association, more than five million Americans are living with heart failure, with about 670,000 new cases diagnosed every year. People with heart failure often have shortness of breath and fatigue. Years of living with blocked arteries and/or high blood pressure can leave a heart too weak to pump enough blood to the body. As symptoms worsen, advanced heart failure develops.

A patient suffering from heart failure may use a VAD while awaiting a heart transplant or as a long term destination therapy. A patient may also use a VAD while recovering from heart surgery. Thus, a VAD can supplement a weak heart (i.e., partial support) or can effectively replace the natural heart's function.

### BRIEF SUMMARY

The following presents a simplified summary of some systems and methods of the present disclosure in order to provide a basic understanding of the systems and methods of the present disclosure. This summary is not an extensive overview of the systems and methods of the present disclosure. It is not intended to identify key/critical elements of the systems and methods of the present disclosure or to delineate the scope of the systems and methods of the present disclosure. Its sole purpose is to present some systems and methods of the present disclosure in a simplified form as a prelude to the more detailed description that is presented later.

The present disclosure describes systems and methods for remotely managing the care of a patient with an implanted ventricular assist device. A patient can have an implanted pulmonary artery pressure sensor via which pulmonary artery pressures of the patient are measured. The measured pulmonary artery pressures and measured operational parameters of the ventricular assist device are remotely accessible by a physician or clinician and can be monitored by the system as specified by the physician or clinician to detect changes in the hemodynamic state of the patient for which updates to the control setting of the VAD and/or updates to one or more medical prescriptions of the patient may be appropriate. The systems and methods described herein improve patient care by enabling increased patient monitoring frequency coupled with increased data on the patient's hemodynamic state to enable more informed patient management decisions.

Thus, in one aspect, a system for managing care of a patient having an implanted left ventricular assist device (LVAD) and an implanted pulmonary artery pressure (PAP) sensor includes a patient management server and a clinician portal. The patient management server is configured to receive PAP data and LVAD data for a patient. The PAP data is indicative of PAP of the patient measured via a PAP sensor implanted in the patient. The LVAD data is indicative of measured operational parameters of an LVAD implanted in the patient. The clinician portal is configured to be communicatively coupled with the patient management server. The clinician portal is configured to communicate an alert to a clinician in response to at least one of the PAP data and the LVAD data being indicative of a change in a hemodynamic condition of the patient.

The PAP of the patient can be measured intermittently over time so that the PAP data is indicative of a trend of the PAP of the patient over time. The PAP data can be indicative of pulmonary artery diastolic pressures (PADPs) of the patient, pulmonary artery mean pressures (PAMPs) of the patient, and heart rates of the patient.

The PAP of the patient can be monitored to detect a change in the hemodynamic state of the patient. For example, the alert can be communicated to the clinician in response to the PAP of the patient being outside a PAP normal operating range established by the clinician for the patient.

The LVAD data can be indicative of blood flow related aspects of the LVAD. For example, the LVAD data can be indicative of at least two of: (a) a minimum flow rate of blood through the LVAD over each of at least one cardiac cycle of the patient, (b) an average flow rate of blood through the LVAD over each of the at least one cardiac cycle of the patient, or (c) a magnitude of variation of flow rate of blood through the LVAD within each of the at least one cardiac cycle of the patient. The LVAD data can be further indicative of a frequency of suction events of the LVAD.

In some circumstances, the alert can include a message to the clinician that specifies a possible condition of the patient and/or the LVAD. For example, the alert can include a message to the clinician that a rotational speed of the LVAD may be too high and/or the patient may be hypovolemic, in response to the PAP of the patient being below the PAP normal operating range for the patient and the frequency of suction events of the LVAD being above a normal range of the frequency of suction events of the LVAD. As another example, the alert can include a message that informs the clinician of a possible occlusion of the LVAD; the alert can be triggered in response to the PAP being above the PAP normal operating range for the patient, the average flow rate of blood through the LVAD being below a normal operating range of the average flow rate of blood through the LVAD for the patient, and the magnitude of variation of flow rate of blood through the LVAD being below a normal operating range of the magnitude of variation of flow rate of blood through the LVAD for the patient. In another example, the alert can include a message that informs the clinician that the patient may be hypertensive and/or hypervolemic in response to the PAP being above the PAP normal operating range for the patient, the average flow rate of blood through the LVAD being below a normal operating range of the average flow rate of blood through the LVAD for the patient, and the magnitude of variation of flow rate of blood through the LVAD being above a normal operating range of the magnitude of variation of flow rate of blood through the LVAD for the patient.

The patient management server can further be configured to receive non-invasively measured blood pressure data for the patient indicative of a mean arterial pressure (MAP) of the patient. Accordingly, in some instances, the alert can include a message that informs the clinician of a possible occlusion of the LVAD in response to the PAP being above the PAP normal operating range for the patient, the average flow rate of blood through the LVAD being below a normal operating range of the average flow rate of blood through the LVAD for the patient, the magnitude of variation of flow rate of blood through the LVAD being below a normal operating range of the magnitude of variation of flow rate of blood through the LVAD for the patient, and the MAP of the patient being below a normal operating range of the MAP of the patient. In some instances, the alert can include a message that informs the clinician that the patient may be hypertensive and/or hypervolemic in response to the PAP being above the PAP normal operating range for the patient, the average flow rate of blood through the LVAD being below a normal operating range of the average flow rate of blood through the LVAD for the patient, the magnitude of variation of flow rate of blood through the LVAD being above a normal operating range of the magnitude of variation of flow rate of blood through the LVAD for the patient, and the MAP of the patient being above a normal operating range of the MAP of the patient.

The LVAD data can be indicative of a blood pressure differential across the LVAD over at least one cardiac cycle of the patient. For example, the LVAD can include one or more sensors configured to generate signals that can be processed to estimate a differential in pressure of the blood pumped between an inlet and an outlet of the LVAD.

Optionally, the clinician can revise one or more control settings of the LVAD via the clinician portal to revise a rate of flow of blood through the LVAD. For example, optionally, the clinician can revise a mean rate of rotation of the LVAD to revise a mean rate of flow of blood through the LVAD.

Optionally, the clinician can program an automated revision of one or more control settings of the LVAD via the clinician portal to revise a rate of flow of blood through the LVAD based on the PAP data and/or the LVAD data. For example, the automated revision of the one or more control settings of the LVAD can increase an average rotational speed of the LVAD in response to an increase in PAP of the patient. As another example, the automated revision of the one or more control settings of the LVAD decrease an average rotational speed of the LVAD in response to an increase in a frequency of suction events of the LVAD. As another example, the automated revision of the one or more control settings of the LVAD increase an average rotational speed of the LVAD in response to a decrease in a mean rate of flow through the LVAD. As another example, the automated revision of the one or more control settings of the LVAD decrease an average rotational speed of the LVAD in response to a decrease in a magnitude of change inflow rate between an average flow rate through the LVAD and a minimum flow rate through the LVAD over a cardiac cycle of the patient.

Optionally, the clinician can update a medication prescription for the patient via the clinician portal in response to review of the PAP data and/or the LVAD data by the clinician. Optionally, the clinician can program an automated update of a medical prescription for the patient based on the PAP data and/or the LVAD data.

In another aspect, a method of managing care of a patient having an implanted left ventricular assist device (LVAD) is provided. The method includes: (a) receiving, by a patient management server, pulmonary artery pressure (PAP) data for the patient indicative of pulmonary artery pressures of the patient measured via a PAP sensor implanted in the patient; (b) receiving, by the patient management server, LVAD data for the patient indicative of measured operational parameters of an LVAD implanted in the patient; and (c) communicating, via a clinician portal, an alert to a clinician in response to at least one of the PAP data and the LVAD data being indicative of a change in a hemodynamic condition of the patient.

For a fuller understanding of the nature and advantages of the systems and methods of the present disclosure, reference should be made to the ensuing detailed description and accompanying drawings.

### BRIEF DESCRIPTION OF THE DRAWINGS

FIG. 1 shows a simplified schematic diagram of a system for managing care of a patient with an implanted left ventricular assist device, in accordance with aspects of the present disclosure.

FIG. 2 shows a plot of pulmonary artery pressure measured via an implanted pulmonary artery pressure sensor that can be used in the system of FIG. 1.

FIG. 3 shows a plot of pulmonary artery pressure over time that illustrates triggering of an alert in the system of FIG. 1 in response to the pulmonary artery pressure exceeding an upper limit, in accordance with aspects of the present disclosure.

### DETAILED DESCRIPTION

In the following description, various systems and methods of the present disclosure will be described. For purposes of explanation, specific configurations and details are set forth in order to provide a thorough understanding of the systems and methods. However, it will also be apparent to one skilled in the art that the systems and methods of the present disclosure may be practiced without the specific details. Furthermore, well-known features may be omitted or simplified in order not to obscure the system or method being described.

An implanted pulmonary artery pressure (PAP) sensor can be employed to measure pulmonary artery pressures of a patient having an implanted left ventricular assist device (LVAD). The measured pulmonary artery pressures and LVAD data indicative of measured operational parameters of the LVAD are communicated to a patient management server is employed to enable remote management of control settings of the LVAD and/or management of patient medical prescriptions by a clinician and/or algorithms.

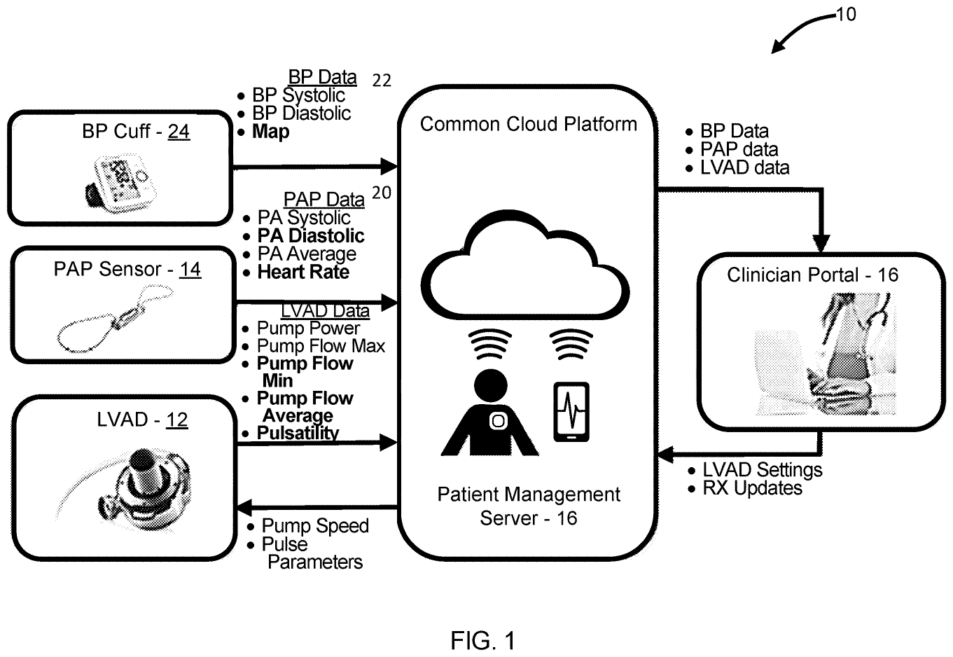

Referring now to the drawings, in which like reference numerals represent like parts throughout the several views, FIG. 1 illustrates a system 10 for managing care of a patient with a left ventricular assist device (LVAD) 12 implanted in the patient and a pulmonary artery pressure (PAP) sensor 14 implanted in the patient. The system 10 includes a patient management server 16 and a clinician portal 18. The patient management server 16 receives LVAD data 18 from the LVAD 12 and pulmonary artery pressure (PAP) data 20 from the PAP sensor 14. Optionally, the patient management server 16 can further receive blood pressure (BP) data 22 from a non-invasive blood pressure cuff 14. The clinician portal 18 enables monitoring of the LVAD data 18, the PAP data 20, and/or the BP data 22 by a clinician for use in updating control settings for the LVAD 12 and/or for updating one or more medical prescriptions for the patient. Optionally, the patient management server 16 communicates the updated control settings to the LVAD 12 and communicates the updated one or medical prescriptions to the patient.

The LVAD data 18 can be measured over a suitable operational time period of the LVAD 12 and is indicative of the instantaneous power used to drive the LVAD 12 over the operational time period, the variation in the maximum flow rate of blood through the LVAD 12 for each cardiac cycle of the patient during the operational time period, the variation in the minimum flow rate of blood through the LVAD 12 for each cardiac cycle of the patient during the operational time period, the variation in the average flow rate of blood through the LVAD 12 during each cardiac cycle of the patient during the operational time period, and the pulsatility of the LVAD 12 (which can be defines as the maximum flow rate of blood through the LVAD 12 minus the minimum flow rate of blood through the LVAD 12 over a cardiac cycle of the patient, or alternatively as the mean flow rate of blood through the LVAD 12 minus the minimum flow rate of blood through the LVAD 12 over a cardiac cycle of the patient). Optionally, the LVAD data 18 can further includes time of suction events and/or frequency of suction events for the LVAD 12.

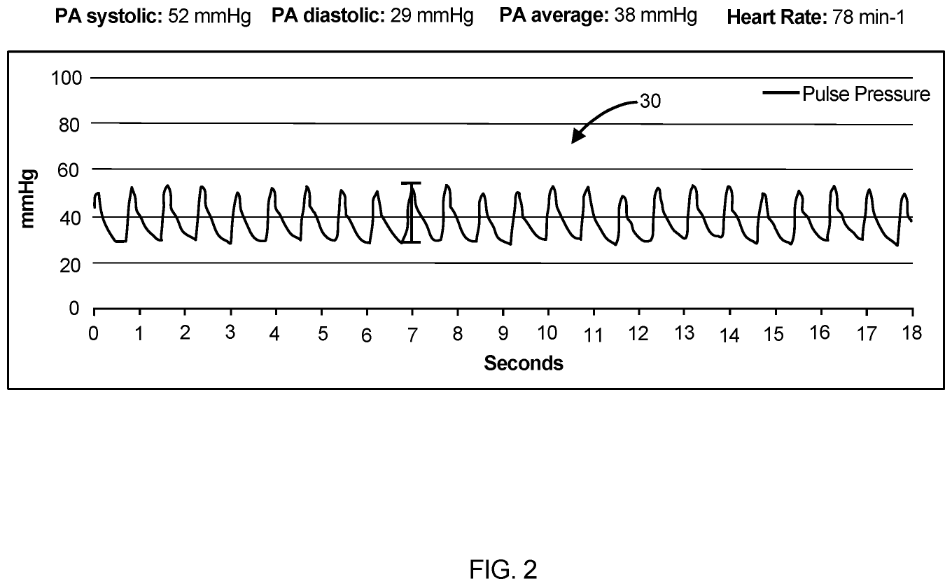

FIG. 2 shows a plot of pulmonary artery pressures 30 measured via the PAP sensor 14 over a suitable operational time period of the PAP sensor 14. The PAP sensor 14 can be configured to be interrogated via an interrogation signal during the operational time period of the PAP sensor 14 to power measurement of the pulmonary artery pressures 30 by the PAP sensor 14. Accordingly, in many instances, the pulmonary artery pressures 30 are intermittently measured via the PAP sensor 14 (e.g., once a day over a limited time span). The system 10 can be configured to process the pulmonary artery pressures 30 to determine components of the PAP data 20 including a pulmonary artery systolic pressure, a pulmonary artery diastolic pressure, a pulmonary artery average pressure, and a heart rate of the patient for each of the operational time periods of the PAP sensor 14.

### Patient Hemodynamic and LVAD Operational Parameter Monitoring

In one aspect, the system 10 provides for remote historical display/trending of patient hemodynamic parameters and LVAD operational parameter monitoring by clinical staff responsible for managing the patient, the LVAD 12, and the PAP sensor 14. The parameters that can be displayed and/or monitored include: 1) MAP (mmHg) (BP Cuff), 2) PA Systolic (mmHg) (PAP Sensor), 3) PA Diastolic (mmHg) (PAP Sensor), 4) PA Average (mmHg) (PAP Sensor), 5) Heart Rate (BPM) (PAP Sensor), 6) Pump Power (W) (LVAD), 7) Flow Average (L/min) (LVAD), 8) Flow Min (L/min) (LVAD), 9) Flow Max (L/min) (LVAD), 10) Pulsatility (LVAD) (e.g., Flow Max-Flow Min, or Flow Mean-Flow Min) and 11) Suction event frequency (i.e., partial occlusion events) (LVAD).

### Selectable Thresholds for Automatic Notifications

In another aspect, the system 10 can be configured to provide for clinician selectable normal operating ranges and/or thresholds for each of the measured patient hemodynamic parameters and the LVAD 12 operational parameters. The system 10 can be configured to generate and output a notification to the clinician via the clinician portal 16 of the monitored parameter goes outside of the specified normal operating range or exceeds the specified threshold.

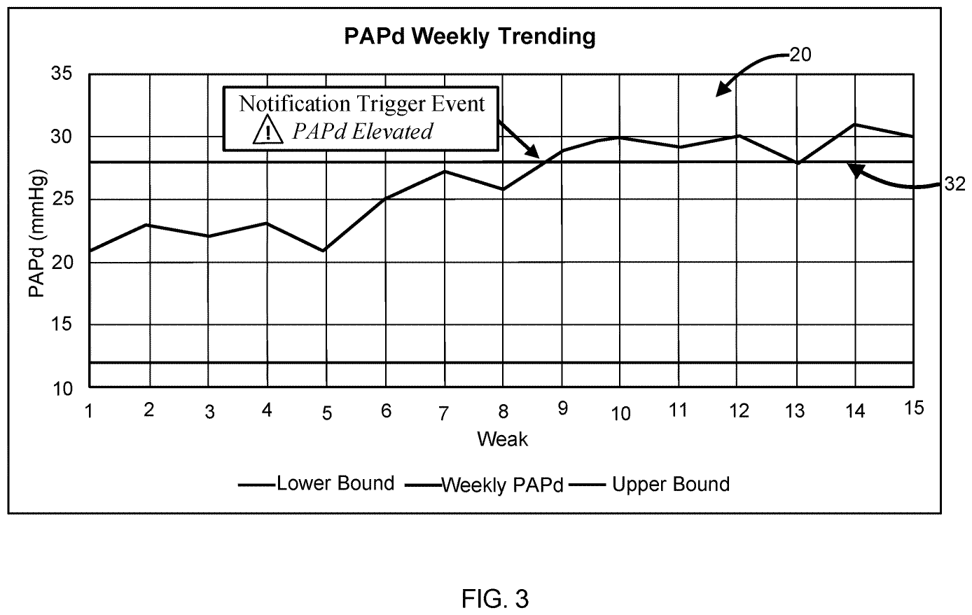

FIG. 3 shows a plot of pulmonary artery diastolic pressure over time that illustrates triggering of an alert in the system of FIG. 1 in response to the pulmonary artery diastolic pressure exceeding an upper limit. Optionally, a clinician can define a normal operating range of any suitable parameter of the PAP data 20 (e.g., PA systolic pressure, PA diastolic pressure, PA average pressure) for use in triggering an alert to the clinician via the clinician portal 16 when the monitored parameter goes outside the designated normal operating range. For example, as illustrated in FIG. 3, a PAPd elevated alert is generated in response to the PA diastolic pressure exceeding a designated upper limit 32 for the PA diastolic pressure.

### Condition Notifications Based on Combined Triggers

In addition to threshold notifications, the system 10 can be configured to provide messages to the clinician specifying possible hemodynamic conditions of the patient consistent with particular combined states of patient hemodynamic parameters and/or the LVAD 12 operations parameters. For example, the system 10 can automatically generate and output a message to the clinician that the patient may be hypertensive and/or hypervolemic when the patient's PA diastolic pressure is high, the average flow through the LVAD 12 is low, the pulsatility of the LVAD 12 is high, and the mean atrial pressure (MAP) of the patient is high. As another example, the system 10 can automatically generate and output a message to the clinician that the pump speed may be too high and/or the patient may be hypovolemic when the patient's PA diastolic pressure is low and the LVAD suction event frequency is high. As another example, the system 10 can automatically generate and output a message to the clinician alerting the clinician of a possible LVAD occlusion in response to the patient's PA diastolic pressure being high, the LVAD average flow being low, the LVAD pulsatility being low, and the MAP being low.

### Remote Updates to LVAD Control Settings and/or Medication Prescriptions

In another aspect, the system 10 can be configured to enable the clinician to update rotational speed settings of the LVAD 12 and medication (RX) updates/recommendations remotely via the clinician portal 16. The rotational speed settings of the LVAD 12 can be adjusted by a controller of the LVAD 12 following a wireless sync with the patient management server 16.

### Automated Closed-Loop Control of the LVAD

In another aspect, the system 10 can be configured to provide the clinician with the ability to program in automated (closed loop) rotational speed adjustment of the LVAD 12 based on the any suitable combination of the measured patient hemodynamic parameters described herein and/or the measured operational parameters of the LVAD 12 described herein. The system 10 can be configured to enable the clinician to customize control of the LVAD 12 according to specific patient needs/goals. For example, the system 10 can be configured to enable the clinician to program in closed loop rotational speed adjustment of the LVAD 12 in which: 1) the rotational speed of the LVAD 12 is increased as diastolic pulmonary pressure increases, 2) the rotational speed of the LVAD 12 is decreased as suction event frequency increases, 3) the rotational speed of the LVAD 12 is increased as mean flow decreases, and/or 4) the rotational speed of the LVAD 12 is decreased as flow pulsatility decreases. The relative “gain” of VAD speed to parameter change can be set by the clinician. Overall upper and lower VAD speed limits can be set by the clinical to ensure safe overall speed ranges regardless of algorithm decisions (speed limits).

Optionally, the control settings for the LVAD 12 that can be updated can include a nominal rotational speed (rotations/minute) of an impeller of the LVAD 12 and a low speed limit (rotations/minute) of the impeller. In some systems and methods in which the LVAD 12 can be operated in an artificial pulse mode to generate blood pressure pulses similar to blood pressure pulses generated by a native heart, the control settings for the LVAD 12 can further include suitable pulse parameters (e.g., pulse amplitude (rotations/minute), pulse duration (ms)).

Other variations are within the spirit of the systems and methods of the present disclosure. Thus, while the systems and methods of the present disclosure are susceptible to various modifications and alternative constructions, certain illustrated systems and methods thereof are shown in the drawings and have been described above in detail. It should be understood, however, that there is no intention to limit the systems and methods of the present disclosure to the specific form or forms disclosed, but on the contrary, the systems and methods of the present disclosure are to cover all modifications, alternative constructions, and equivalents falling within the spirit and scope of the present disclosure, as defined in the appended claims.

The use of the terms “a” and “an” and “the” and similar referents in the context of describing the systems and methods of the present disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the systems and methods of the present disclosure and does not pose a limitation on the scope of the systems and methods of the present disclosure unless otherwise claimed. No language in the specification should be construed as indicating any non-claimed element as essential to the practice of the systems and methods of the present disclosure.

Preferred systems and methods according to the present disclosure are described herein, including the best mode known to the inventors for carrying out the systems and methods of the present disclosure. Variations of those preferred systems and methods may become apparent to those of ordinary skill in the art upon reading the foregoing description. The inventors expect skilled artisans to employ such variations as appropriate, and the inventors intend for the systems and methods of the present disclosure to be practiced otherwise than as specifically described herein. Accordingly, the systems and methods of the present disclosure include all modifications and equivalents of the subject matter recited in the claims appended hereto as permitted by applicable law. Moreover, any combination of the above-described elements in all possible variations thereof is encompassed by the systems and methods of the present disclosure unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

The following relate to numbered aspects of the invention:
1. A system for managing care of a patient having an implanted left ventricular assist device (LVAD) and an implanted pulmonary artery pressure (PAP) sensor, the system comprising:
   a patient management server configured to receive PAP data and LVAD data for a patient, wherein the PAP data is indicative of PAP of the patient measured via a PAP sensor implanted in the patient, and wherein the LVAD data is indicative of measured operational parameters of an LVAD implanted in the patient; and
   a clinician portal configured to be communicatively coupled with the patient management server, wherein the clinician portal is configured to communicate an alert to a clinician in response to at least one of the PAP data and the LVAD data being indicative of a change in a hemodynamic condition of the patient.
2. The system of aspect 1, wherein the PAP of the patient is measured intermittently over time so that the PAP data is indicative of a trend of the PAP of the patient over time.
3. The system of aspect 1 or 2, wherein the alert is communicated to the clinician in response to the PAP of the patient being outside a PAP normal operating range established by the clinician for the patient.
4. The system of any one of aspects 1 to 3, wherein the LVAD data is indicative of at least two of:
   a minimum flow rate of blood through the LVAD over each of at least one cardiac cycle of the patient;
   an average flow rate of blood through the LVAD over each of the at least one cardiac cycle of the patient; or
   a magnitude of variation of flow rate of blood through the LVAD within each of the at least one cardiac cycle of the patient.
5. The system of aspect 4, wherein:
   the LVAD data is indicative a frequency of suction events of the LVAD; and
   the alert comprises a message to the clinician that a rotational speed of the LVAD may be too high and/or the patient may be hypovolemic, in response to the PAP of the patient being below the PAP normal operating range for the patient and/or the frequency of suction events of the LVAD being above a normal range of the frequency of suction events of the LVAD.
6. The system of aspect 4, wherein:
   the alert comprises a message that informs the clinician of a possible occlusion of the LVAD; and
   the alert is triggered in response to the PAP being above the PAP normal operating range for the patient, the average flow rate of blood through the LVAD being below a normal operating range of the average flow rate of blood through the LVAD for the patient, and the magnitude of variation of flow rate of blood through the LVAD being below a normal operating range of the magnitude of variation of flow rate of blood through the LVAD for the patient.
   7. The system of aspect 4, wherein the alert comprises a message that informs the clinician that the patient may be hypertensive and/or hypervolemic in response to the PAP being above the PAP normal operating range for the patient, the average flow rate of blood through the LVAD being below a normal operating range of the average flow rate of blood through the LVAD for the patient, and the magnitude of variation of flow rate of blood through the LVAD being above a normal operating range of the magnitude of variation of flow rate of blood through the LVAD for the patient.
8. The system of aspect 4, wherein:
   the patient management server is further configured to receive non-invasively measured blood pressure data for the patient indicative of a mean arterial pressure (MAP) of the patient; and
   the alert comprises a message that informs the clinician of a possible occlusion of the LVAD in response to the PAP being above the PAP normal operating range for the patient, the average flow rate of blood through the LVAD being below a normal operating range of the average flow rate of blood through the LVAD for the patient, the magnitude of variation of flow rate of blood through the LVAD being below a normal operating range of the magnitude of variation of flow rate of blood through the LVAD for the patient, and the MAP of the patient being below a normal operating range of the MAP of the patient.
9. The system of aspect 4, wherein:
   the patient management server is further configured to receive non-invasively measured blood pressure data for the patient indicative of a mean arterial pressure (MAP) of the patient; and
   the alert comprises a message that informs the clinician that the patient may be hypertensive and/or hypervolemic in response to the PAP being above the PAP normal operating range for the patient, the average flow rate of blood through the LVAD being below a normal operating range of the average flow rate of blood through the LVAD for the patient, the magnitude of variation of flow rate of blood through the LVAD being above a normal operating range of the magnitude of variation of flow rate of blood through the LVAD for the patient, and the MAP of the patient being above a normal operating range of the MAP of the patient.
10. The system of aspect 4, wherein the LVAD data is indicative of a blood pressure differential across the LVAD over at least one cardiac cycle of the patient.
11. The system of any one of aspects 1 to 10, wherein the clinician can revise one or more control settings of the LVAD via the clinician portal to revise a rate of flow of blood through the LVAD.
12. The system of any one of aspects 1 to 10, wherein the clinician can program an automated revision of one or more control settings of the LVAD via the clinician portal to revise a rate of flow of blood through the LVAD based on the PAP data and/or the LVAD data.
13. The system of aspect 12, wherein the automated revision of the one or more control settings of the LVAD increase an average rotational speed of the LVAD in response to an increase in PAP of the patient.
14. The system of aspect 12, wherein the automated revision of the one or more control settings of the LVAD decrease an average rotational speed of the LVAD in response to an increase in a frequency of suction events of the LVAD.
15. The system of aspect 12, wherein the automated revision of the one or more control settings of the LVAD increase an average rotational speed of the LVAD in response to a decrease in a mean rate of flow through the LVAD.
16. The system of aspect 12, wherein the automated revision of the one or more control settings of the LVAD decrease an average rotational speed of the LVAD in response to a decrease in a magnitude of change inflow rate between an average flow rate through the LVAD and a minimum flow rate through the LVAD over a cardiac cycle of the patient.
17. The system of any one of aspects 1 to 10, wherein the clinician can update a medication prescription for the patient via the clinician portal in response to review of the PAP data and/or the LVAD data by the clinician.
18. The system of any one of aspects 1 to 10, wherein the clinician can program an automated update of a medical prescription for the patient based on the PAP data and/or the LVAD data.
19. A method of managing care of a patient having an implanted left ventricular assist device (LVAD), the method comprising:
   receiving, by a patient management server, pulmonary artery pressure (PAP) data for the patient indicative of pulmonary artery pressures of the patient measured via a PAP sensor implanted in the patient;
   receiving, by the patient management server, LVAD data for the patient indicative of measured operational parameters of an LVAD implanted in the patient; and
   communicating, via a clinician portal, an alert to a clinician in response to at least one of the PAP data and the LVAD data being indicative of a change in a hemodynamic condition of the patient.
20. The method of aspect 19, wherein the PAP of the patient is measured intermittently over time so that the PAP data is indicative of a trend of the PAPs of the patient over time.
21. The method of aspect 19 or 20, wherein the alert is communicated to the clinician in response to the PAP of the patient being outside a PAP normal operating range established by the clinician for the patient.
22. The method of any one of aspects 19 to 21, wherein the LVAD data is indicative of at least two of:
   a minimum flow rate of blood through the LVAD over each of at least one cardiac cycle of the patient;
   an average flow rate of blood through the LVAD over each of the at least one cardiac cycle of the patient; or
   a magnitude of variation of flow rate of blood through the LVAD within each of the at least one cardiac cycle of the patient.
23. The method of aspect 22, wherein:
   the LVAD data is indicative a frequency of suction events of the LVAD; and
   the alert comprises a message to the clinician that a rotational speed of the LVAD may be too high and/or the patient may be hypovolemic, in response to the PAP of the patient being below the PAP normal operating range for the patient and the frequency of suction events of the LVAD being above a normal range of the frequency of suction events of the LVAD.
24. The method of aspect 22, wherein the alert comprises a message that informs the clinician of a possible occlusion of the LVAD in response to the PAP being above the PAP normal operating range for the patient, the average flow rate of blood through the LVAD being below a normal operating range of the average flow rate of blood through the LVAD for the patient, and the magnitude of variation of flow rate of blood through the LVAD being below a normal operating range of the magnitude of variation of flow rate of blood through the LVAD for the patient.
25. The method of claim aspect 22, wherein the alert comprises a message that informs the clinician that the patient may be hypertensive and/or hypervolemic in response to the PAP being above the PAP normal operating range for the patient, the average flow rate of blood through the LVAD being below a normal operating range of the average flow rate of blood through the LVAD for the patient, and the magnitude of variation of flow rate of blood through the LVAD being above a normal operating range of the magnitude of variation of flow rate of blood through the LVAD for the patient.
26. The method of aspect 22, further comprising receiving, by the patient management server, non-invasively measured blood pressure data for the patient indicative of a mean arterial pressure (MAP) of the patient, and wherein the alert comprises a message that informs the clinician of a possible occlusion of the LVAD in response to the PAP being above the PAP normal operating range for the patient, the average flow rate of blood through the LVAD being below a normal operating range of the average flow rate of blood through the LVAD for the patient, the magnitude of variation of flow rate of blood through the LVAD being below a normal operating range of the magnitude of variation of flow rate of blood through the LVAD for the patient, and the MAP of the patient being below a normal operating range of the MAP of the patient.
27. The method of aspect 22, further comprising receiving, by the patient management server, non-invasively measured blood pressure data for the patient indicative of a mean arterial pressure (MAP) of the patient, and wherein the alert comprises a message that informs the clinician that the patient may be hypertensive and/or hypervolemic in response to the PAP being above the PAP normal operating range for the patient, the average flow rate of blood through the LVAD being below a normal operating range of the average flow rate of blood through the LVAD for the patient, the magnitude of variation of flow rate of blood through the LVAD being above a normal operating range of the magnitude of variation of flow rate of blood through the LVAD for the patient, and the MAP of the patient being above a normal operating range of the MAP of the patient.
28. The method of aspect 22, wherein the LVAD data is indicative of a blood pressure differential across the LVAD over at least one cardiac cycle of the patient.
29. The method of any one of aspects 19 to 28, further comprising revising, via the clinician portal, one or more control settings of the LVAD to revise a rate of flow of blood through the LVAD.
30. The method of any one of aspects 19 to 28, further comprising programming, via the clinician portal, an automated revision of one or more control settings of the LVAD to revise a rate of flow of blood through the LVAD based on the PAP data and/or the LVAD data.
31. The method of aspect 30, wherein the automated revision of the one or more control settings of the LVAD increase an average rotational speed of the LVAD in response to an increase in PAP of the patient.
32. The method of aspect 30, wherein the automated revision of the one or more control settings of the LVAD decrease an average rotational speed of the LVAD in response to an increase in a frequency of suction events of the LVAD.
33. The method of aspect 30, wherein the automated revision of the one or more control settings of the LVAD increase an average rotational speed of the LVAD in response to a decrease in a mean rate of flow through the LVAD.
34. The method of aspect 30, wherein the automated revision of the one or more control settings of the LVAD decrease an average rotational speed of the LVAD in response to a decrease in a magnitude of change inflow rate between an average flow rate through the LVAD and a minimum flow rate through the LVAD over a cardiac cycle of the patient.
35. The method of any one of aspects 19 to 34, further comprising updating, via the clinician portal, a medication prescription for the patient in response to review of the PAP data and/or the LVAD data by the clinician.
36. The method of any one of aspects 19 to 34, further comprising programming, via the clinician portal, an automated update of a medical prescription for the patient based on the PAP data and/or the LVAD data.

1. A system for managing care of a patient having an implanted left ventricular assist device (LVAD) and an implanted pulmonary artery pressure (PAP) sensor, the system comprising:
a patient management server configured to receive PAP data and LVAD data for a patient, wherein the PAP data is indicative of PAP of the patient measured via a PAP sensor implanted in the patient, and wherein the LVAD data is indicative of measured operational parameters of an LVAD implanted in the patient; and
a clinician portal configured to be communicatively coupled with the patient management server, wherein the clinician portal is configured to communicate an alert to a clinician in response to at least one of the PAP data and the LVAD data being indicative of a change in a hemodynamic condition of the patient.

2. The system of claim 1, wherein the PAP of the patient is measured intermittently over time so that the PAP data is indicative of a trend of the PAP of the patient over time.

3. The system of claim 2, wherein the alert is communicated to the clinician in response to the PAP of the patient being outside a PAP normal operating range established by the clinician for the patient.

4. The system of claim 3, wherein the LVAD data is indicative of at least two of:
a minimum flow rate of blood through the LVAD over each of at least one cardiac cycle of the patient;
an average flow rate of blood through the LVAD over each of the at least one cardiac cycle of the patient; or
a magnitude of variation of flow rate of blood through the LVAD within each of the at least one cardiac cycle of the patient.

5. The system of claim 4, wherein:
the LVAD data is indicative a frequency of suction events of the LVAD; and
the alert comprises a message to the clinician that a rotational speed of the LVAD may be too high and/or the patient may be hypovolemic, in response to the PAP of the patient being below the PAP normal operating range for the patient and/or the frequency of suction events of the LVAD being above a normal range of the frequency of suction events of the LVAD.

6. The system of claim 4, wherein:
the alert comprises a message that informs the clinician of a possible occlusion of the LVAD; and
the alert is triggered in response to the PAP being above the PAP normal operating range for the patient, the average flow rate of blood through the LVAD being below a normal operating range of the average flow rate of blood through the LVAD for the patient, and the magnitude of variation of flow rate of blood through the LVAD being below a normal operating range of the magnitude of variation of flow rate of blood through the LVAD for the patient.

7. The system of claim 4, wherein the alert comprises a message that informs the clinician that the patient may be hypertensive and/or hypervolemic in response to the PAP being above the PAP normal operating range for the patient, the average flow rate of blood through the LVAD being below a normal operating range of the average flow rate of blood through the LVAD for the patient, and the magnitude of variation of flow rate of blood through the LVAD being above a normal operating range of the magnitude of variation of flow rate of blood through the LVAD for the patient.

8. The system of claim 4, wherein:
the patient management server is further configured to receive non-invasively measured blood pressure data for the patient indicative of a mean arterial pressure (MAP) of the patient; and
the alert comprises a message that informs the clinician of a possible occlusion of the LVAD in response to the PAP being above the PAP normal operating range for the patient, the average flow rate of blood through the LVAD being below a normal operating range of the average flow rate of blood through the LVAD for the patient, the magnitude of variation of flow rate of blood through the LVAD being below a normal operating range of the magnitude of variation of flow rate of blood through the LVAD for the patient, and the MAP of the patient being below a normal operating range of the MAP of the patient.

9. The system of claim 4, wherein:
the patient management server is further configured to receive non-invasively measured blood pressure data for the patient indicative of a mean arterial pressure (MAP) of the patient; and
the alert comprises a message that informs the clinician that the patient may be hypertensive and/or hypervolemic in response to the PAP being above the PAP normal operating range for the patient, the average flow rate of blood through the LVAD being below a normal operating range of the average flow rate of blood through the LVAD for the patient, the magnitude of variation of flow rate of blood through the LVAD being above a normal operating range of the magnitude of variation of flow rate of blood through the LVAD for the patient, and the MAP of the patient being above a normal operating range of the MAP of the patient.

10. The system of claim 4, wherein the LVAD data is indicative of a blood pressure differential across the LVAD over at least one cardiac cycle of the patient.

11. The system of claim 1, wherein the clinician can revise one or more control settings of the LVAD via the clinician portal to revise a rate of flow of blood through the LVAD.

12. The system of claim 1, wherein the clinician can program an automated revision of one or more control settings of the LVAD via the clinician portal to revise a rate of flow of blood through the LVAD based on the PAP data and/or the LVAD data.

13. The system of claim 12, wherein the automated revision of the one or more control settings of the LVAD increase an average rotational speed of the LVAD in response to an increase in PAP of the patient.

14. The system of claim 12, wherein the automated revision of the one or more control settings of the LVAD decrease an average rotational speed of the LVAD in response to an increase in a frequency of suction events of the LVAD.

15. The system of claim 12, wherein the automated revision of the one or more control settings of the LVAD increase an average rotational speed of the LVAD in response to a decrease in a mean rate of flow through the LVAD.

16. The system of claim 12, wherein the automated revision of the one or more control settings of the LVAD decrease an average rotational speed of the LVAD in response to a decrease in a magnitude of change inflow rate between an average flow rate through the LVAD and a minimum flow rate through the LVAD over a cardiac cycle of the patient.

17. The system of claim 1, wherein the clinician can update a medication prescription for the patient via the clinician portal in response to review of the PAP data and/or the LVAD data by the clinician.

18. The system of claim 1, wherein the clinician can program an automated update of a medical prescription for the patient based on the PAP data and/or the LVAD data.

19.-36. (canceled)
